(19) United States
(12) Patent Application Publication
(10) Pub. No.: US 2018/0235912 A1
(43) Pub. Date: Aug. 23, 2018

(54) PHARMACEUTICAL COMPOSITION FOR PREVENTING OR TREATING AUTOIMMUNE DISEASES COMPRISING THIOUREA DERIVATIVE

(71) Applicant: SEOUL NATIONAL UNIVERSITY R & DB FOUNDATION, Seoul (KR)

(72) Inventors: Mi Ock LEE, Seoul (KR); Hyeung Geun PARK, Seoul (KR); Mi-La CHO, Seoul (KR); Yong-Hyun HAN, Seoul (KR); Hyeon-Ji KIM, Seoul (KR); Jin-Sil PARK, Seoul (KR)

(73) Assignee: SEOUL NATIONAL UNIVERSITY R & DB FOUNDATION, Seoul (KR)

(21) Appl. No.: 15/752,079

(22) PCT Filed: Aug. 12, 2016
(86) PCT No.: PCT/KR2016/008867

§ 371 Date: Feb. 12, 2018

(30) Foreign Application Priority Data
Aug. 12, 2015 (KR) 10-2015-0113739
Aug. 11, 2016 (KR) 10-2016-0102224

(51) Int. Cl. A61K 31/17; A61P 1/00; A61P 3/10; A61P 17/06; A61P 19/02

(52) U.S. Cl. CPC A61K 31/17; A61P 1/00; A61P 19/02; A61P 17/06; A61P 3/10

(57) ABSTRACT

The present invention relates to a novel use of a thiourea derivative and, more specifically, to a pharmaceutical composition for preventing or treating autoimmune diseases comprising a thiourea derivative as an active ingredient. The thiourea derivative according to the present invention can inhibit the transcription of inflammatory genes such as TNF-α, IL-1β, NOS2 and IL-6, and also can inhibit the activity or production of Th17 and increase the activity or production of a regulatory T cell (Treg). Thus, it is expected that the thiourea derivative may be usefully used in a pharmaceutical composition, a health food composition, etc. for the prevention, improvement or treatment of various autoimmune diseases including rheumatoid arthritis.

### TECHNICAL FIELD

The present invention relates to a novel use of a thiourea derivative and, more specifically, to a pharmaceutical composition for preventing or treating autoimmune diseases including a thiourea derivative as an active ingredient.

### BACKGROUND ART

In humans, the immune system acts to protect the body from foreign antigens that invade the human body, but do not invade their own tissues due to self-tolerance. However, when the self-tolerance of the immune system is destroyed so that immune cells recognize proteins normally expressed by their own genes as a target of attack to produce antibodies or cause T cell responses to destroy normal tissues, this is referred to an autoimmune reaction. When specific symptoms develop, they are referred to autoimmune diseases.

As one of such autoimmune diseases, rheumatoid arthritis is a chronic disease that causes inflammation in the synovium surrounding the joint to spread it into the cartilage and bone around the synovium, thereby destroying the joint and causing physical disabilities. As inflammation appears in the synovium inside the joint and immune cells are gathered in blood, an increase in joint fluid causes swollen joints, accompanied by pain in the joints. Rheumatoid arthritis is generally a disease caused by autoimmune dysfunction, and an abnormal immune function causes inflammation. In this case, T cells, B cells and macrophages are generally involved in the inflammatory response in the joints, and inflammation is induced by TNF-α, interleukin (IL)-1β, IL-6, and IL-17 secreted by these cells. Particularly, it has been known that M1 macrophages secreting TNF-α and IL-1β and Th17 cells secreting IL-17 further aggravate diseases such as rheumatoid arthritis.

In recent years, the treatment of autoimmune diseases including rheumatoid arthritis is mainly achieved using TNF inhibitors (Infliximab, Etanercept, and the like), IL-1 inhibitors (Anakinra, Canakinumab), steroidal or non-steroid anti-inflammatory agents (NSAID), cytokine inhibitors (Actemra), signal transduction inhibitors (JAK3 inhibitors), or TNF-α-associated antibody-therapeutic agents. However, the use of such therapeutic agents causes side effects such as itching, respiratory infections, and the like, and focuses on the relief of pain through inflammation control. Therefore, because it is difficult to cure autoimmune diseases completely, there is no fundamental therapy against the autoimmune disease.

As described above, most autoimmune diseases have no clear causes and pathogenesis and are difficult to treat because there are a wide range of autoimmune diseases. The therapeutic agents currently used have problems in that patients develop resistance to the therapeutic agents or cause severe side effects when the patients take the therapeutic agents for a long time. Therefore, there is an urgent demand for development of novel therapeutic agents. In this regard, studies on the novel therapeutic agents have been conducted (Korean Patent Unexamined Publication No. 10-2013-0031229, and the like), but the results of the studies still remain insignificant.

### DISCLOSURE

### Technical Problem

The present invention is designed to solve the problems of the prior art. Therefore, the present inventors have found that a thiourea derivative, which is a RORα activator, inhibits an inflammatory response and hinders the differentiation and activation of Th17 cells, and simultaneously promotes the production of Treg cells. Therefore, the present invention has been completed based on these facts.

Therefore, it is an object of the present invention to provide a pharmaceutical composition for preventing or treating autoimmune diseases, which includes a thiourea derivative or a pharmaceutically acceptable salt thereof as an active ingredient.

However, these objects of the present invention are not limited to the objects as described above. Therefore, it should be understood that the other objects of the present invention which are not described herein will become clearly apparent from the following description.

### Technical Solution

To achieve the objects of the present invention, one aspect of the present invention provides a pharmaceutical composition for preventing or treating autoimmune diseases, which includes a compound represented by the following Formula 1 or a pharmaceutically acceptable salt thereof as an active ingredient:

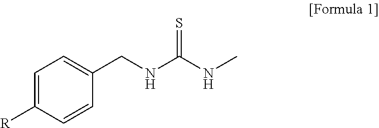

wherein R represents a phenoxy group, a benzyloxy group, or a pyridinyl group.

According to one exemplary embodiment of the present invention, the autoimmune diseases may be selected from the group consisting of rheumatoid arthritis, psoriatic arthritis, psoriasis, systemic scleroderma, sclerosis, multiple sclerosis, inflammatory bowel disease, systemic lupus erythematosus, Crohn's disease, septicemia, and type I diabetes.

According to another exemplary embodiment of the present invention, the composition may inhibit the production of Th17 cells.

According to still another exemplary embodiment of the present invention, the composition may promote the production of regulatory T cells (Tregs).

According to yet another exemplary embodiment of the present invention, the composition may inhibit the expression of a TNF-α, NOS2, IL-1β or IL-6 inflammatory gene to inhibit the differentiation of M1 macrophages.

According to yet another exemplary embodiment of the present invention, the composition may promote the expression of an anti-inflammatory gene such as IL-10, Arg1, Retnla or CD206 in the macrophages to enhance the differentiation of M2 macrophages.

Another aspect of the present invention provides a method of treating autoimmune diseases, which includes administering a compound represented by Formula 1 or a pharmaceutically acceptable salt thereof to a subject.

Still another aspect of the present invention provides a use of the compound represented by the Formula 1 or the pharmaceutically acceptable salt thereof for treating autoimmune diseases.

### Advantageous Effects

A thiourea derivative according to the present invention can inhibit the transcriptions of inflammatory genes such as TNF-α, IL-1β, NOS2, IL-6, and the like, promote the transcription of anti-inflammatory genes such as IL-10, Arg1, CD206, and the like, and simultaneously inhibit the activation or production of Th17 cells and increase the activation or production of regulatory T cells (Tregs). Therefore, the thiourea derivative according to the present invention can be expected to be effectively used in a pharmaceutical composition, a health food composition, and the like for preventing, improving or treating various autoimmune diseases including rheumatoid arthritis.

### DESCRIPTION OF DRAWINGS

FIG. 1 shows the results of confirming, through real-time PCR, an inhibitory effect of JC1-40 on the transcription of M1 inflammatory genes in a macrophage cell line.

FIGS. 2A and 2B show the results of confirming, through real-time PCR, an inhibitory effect of JC1-40 on the transcription of M1 inflammatory genes in primarily cultured mouse intraperitoneal macrophages.

FIG. 3 shows the results of confirming, through flow cytometry, a decrease in M1-differentiated macrophages in primarily cultured mouse intraperitoneal macrophages when the mouse intraperitoneal macrophages are treated with JC1-40.

FIGS. 4A and 4B show the results of confirming, through real-time PCR, an inhibitory effect of JC1-40 on the transcription of M2 anti-inflammatory genes in primarily cultured mouse intraperitoneal macrophages.

FIG. 5A shows the results of confirming, through real-time PCR, an inhibitory effect of JC1-40 on the transcription of an IL-17 gene in a T cell line.

FIG. 5B shows the results of confirming, through an immunostaining method, an increase in the expression of Tregs by JC1-40 in the T cell line.

FIG. 6A shows the results of confirming, through a flow cytometry staining method, a decrease in Th17 differentiated cells when primarily cultured mouse spleen T cells are treated with a varying concentration of JC1-40.

FIG. 6B shows the results of measuring, through ELISA, an amount of IL-17 in a supernatant when the primarily cultured mouse spleen T cells are treated with a varying concentration of JC1-40.

FIG. 7A shows the results of confirming, through a flow cytometry staining method, a quantitative change of Th17 and Treg differentiated cells when human peripheral blood mononuclear cells (PBMCs) are treated with a varying concentration of JC1-40.

FIG. 7B shows the results of measuring, through ELISA, an amount of IL-17 in a supernatant when the human peripheral blood mononuclear cells (PBMCs) are treated with a varying concentration of JC1-40.

FIGS. 8A and 8B show the results of confirming, through real-time PCR, an inhibitory effect of JC1-40 on the expression of osteoclast differentiation-related factors when an osteoclast precursor which has been differentiated from human peripheral blood mononuclear cells (PBMCs) is treated with a varying concentration of JC1-40.

FIG. 9 shows the results of calculating arthritis incidence and scores in an arthritis animal model (type II collagen-induced arthritis (CIA)) when JC1-40 is administered to the arthritis animal model.

### BEST MODE

The present inventors have endeavored to conduct research on materials which are effective in treating autoimmune diseases including rheumatoid arthritis, and found that a thiourea derivative, which is a RORα activator, inhibits the expression of inflammatory cytokines such as TNF-α, IL-1β, IL-6, and the like, and simultaneously promotes the expression of anti-inflammatory factors such as IL-10, Arg1, CD206, and the like, inhibits or suppresses the differentiation and activation of Th17 cells, and promotes the production of Treg cells. Therefore, the present invention has been completed based on these facts.

Hereinafter, the present invention will be described in detail.

The present invention provides a pharmaceutical composition for preventing or treating autoimmune diseases, which includes a compound represented by the following Formula 1 or a pharmaceutically acceptable salt thereof as an active ingredient.

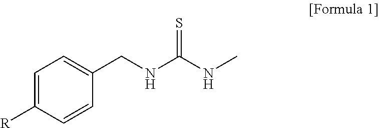

wherein R represents a phenoxy group, a benzyloxy group, or a pyridinyl group.

The term “preventing” or “prevention” used in the present invention refers to all types of actions that inhibit autoimmune diseases or delay the onset of the diseases by administering the pharmaceutical composition according to the present invention.

The term “treating” or “treatment” used in the present invention refers to all types of actions that alleviate or beneficially change the symptoms of autoimmune diseases by administering the pharmaceutical composition according to the present invention.

As a target disease to be improved, prevented or treated by the composition of the present invention, an “autoimmune disease” refers to a disease or disorder that results from a situation in which, when a patient has a problem in inducing or continuously maintaining self-tolerance, an immune response against self-antigens occurs so that their own tissues are attacked. Also, types of the autoimmune disease in the present invention are not limited, but may be selected from the group consisting of rheumatoid arthritis, psoriatic arthritis, psoriasis, systemic scleroderma, sclerosis, multiple sclerosis, inflammatory bowel disease, systemic lupus erythematosus, Crohn's disease, septicemia, and type I diabetes. Preferably, the autoimmune disease may be rheumatoid arthritis.

The compound represented by Formula 1, which is included as the active ingredient in the composition according to the present invention, is a thiourea derivative (referred to as a JC1 compound) using a conventional thiazolidinedione-based compound CGP52608 as a lead compound, and is preferably 1-methyl-3-(4-(pyridin-2-yl)benzyl)-thiourea (JC1-38), 1-(4-benzyloxy-benzyl)-3-methyl-thiourea (JC1-40), or 1-(4-phenoxy-benzyl)-3-methyl-thiourea (JC1-42).

The composition according to the present invention may be effective in treating autoimmune diseases by inhibiting the expression of inflammatory genes to inhibit the differentiation of M1 macrophages.

According to one exemplary embodiment of the present invention, a regulatory effect of JC1-40 on inflammation in a macrophage cell line and primarily cultured mouse intraperitoneal macrophages was examined. As a result, it was confirmed that, when cells are treated with JC1-40, a transcription level of inflammatory cytokines, such as TNF-α, NOS2, IL-1β, and IL-6, which are involved in the induction of inflammation, is significantly reduced and the differentiation of M1 macrophages is suppressed (see Examples 2 and 3).

Also, the composition according to the present invention may have a therapeutic effect on autoimmune diseases by promoting the expression of an IL-10, Arg1, Retnla or CD206 anti-inflammatory gene to enhance the differentiation of M2 macrophages.

According to another exemplary embodiment of the present invention, an inhibitory effect of JC1-40 on the transcription of M1 inflammatory genes in primarily cultured mouse intraperitoneal macrophages was examined. As a result, it was confirmed that, when the cells are treated with JC1-40, a transcription level of IL-10, Arg1, Retnla and CD206, which are involved in anti-inflammatory regulation in an intraperitoneal macrophage cell line, is promoted (see Example 4).

In addition, the composition according to the present invention may have a therapeutic effect on autoimmune diseases by suppressing the activation or production of Th17 cells and increasing the activation or production of regulatory T cells (Tregs).

According to still another exemplary embodiment of the present invention, an inhibitory effect of JC1-40 on Th17 differentiation and IL-17 secretion in primarily cultured mouse spleen T cells and human peripheral blood mononuclear cells (PBMCs) was examined. As a result, it was confirmed that, when the cells are treated with JC1-40, an amount of (IL-17 positive) T cells differentiated into Th17 cells is reduced, whereas an amount of (FOXP3 positive) T cells differentiated into Tregs is increased (see Examples 5, 6, and 7).

Further, it was confirmed that the expression of osteoclast differentiation-related factors is significantly suppressed when an osteoclast precursor, which has been differentiated from human peripheral blood mononuclear cells (PBMCs), is treated with the composition according to the present invention (see Example 8). Moreover, it was confirmed that, when an in vivo arthritis animal model is treated with the composition according to the present invention to observe a course of progression of diseases such as arthritis, the composition according to the present invention has a superior effect of relieving arthritis, compared to a therapeutic agent (Enbrel) used in conventional clinical practice (see Example 9).

The compound represented by Formula 1 according to the present invention may include a pharmaceutically acceptable salt, as well as all types of salts, hydrates, and solvates which may be prepared using conventional methods. When the compound is used in the form of a pharmaceutically acceptable salt, an acid addition salt formed by a pharmaceutically acceptable free acid may be effectively used as the salt. The acid addition salt is obtained from inorganic acids such as hydrochloric acid, nitric acid, phosphoric acid, sulfuric acid, hydrobromic acid, hydroiodic acid, nitrous acid, or phosphorous acid, aliphatic mono- and di-carboxylates, phenyl-substituted alkanoates, hydroxy alkanoates, and alkanedioates, and non-toxic organic acids such as aromatic acids, aliphatic and aromatic sulfonic acids. Such pharmaceutically non-toxic salts include sulfates, pyrosulfates, bisulfates, sulfites, bisulfites, nitrates, phosphates, monohydrogen phosphates, dihydrogen phosphates, metaphosphates, pyrophosphate chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexane-1,6-dioates, benzoates, chlorobenzoates, methylbenzoates, dinitrobenzoates, hydroxybenzoates, methoxybenzoates, phthalates, terephthalates, benzenesulfonates, toluenesulfonates, chlorobenzenesulfonates, xylenesulfonates, phenylacetates, phenylpropionates, phenylbutyrates, citrates, lactates, β-hydroxybutyrates, glycolates, malates, tartrates, methanesulfonates, propanesulfonates, naphthalene-1-sulfonates, naphthalene-2-sulfonates, or mandelates.

The acid addition salt according to the present invention may be prepared using a conventional method, for example prepared by dissolving the compound represented by Formula 1 in an excessive amount of an aqueous acid solution, and precipitating the resulting salt with a water-miscible organic solvent, for example, methanol, ethanol, acetone, or acetonitrile. The acid addition salt may also be prepared by heating an equivalent amount of the compound represented by Formula 1 and an acid or alcohol in water, and then drying the resulting mixture by evaporation or suction-filtering the precipitated salt.

Also, a pharmaceutically acceptable metal salt may be prepared using a base. An alkali metal or alkaline earth metal salt is, for example, obtained by dissolving the compound in an excessive amount of an alkali metal hydroxide or alkaline earth metal hydroxide solution, filtering an undissolved compound salt, and evaporating and drying the filtrate. In this case, the metal salt is preferably prepared using a pharmaceutically suitable sodium, potassium, or calcium salt. Also, a silver salt corresponding to the metal salt is obtained by reacting an alkali metal or alkaline earth metal salt with a suitable silver salt (e.g., silver nitrate).

Meanwhile, the pharmaceutical composition of the present invention may further include one or more known active ingredients having a therapeutic effect on autoimmune diseases in addition to the compound represented by Formula 1 or a pharmaceutically acceptable salt thereof.

The composition of the present invention may further include a suitable carrier, excipient, and diluent, all of which are generally used to prepare the pharmaceutical composition. The carrier, excipient and diluent, which may be included in the composition, include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oils. When the composition is prepared into a formulation, the formulation is prepared using a diluent or excipient generally used in the art, such as a filler, a bulking agent, a binder, a wetting agent, a disintegrating agent, a surfactant, and the like.

The pharmaceutical composition of the present invention may be prepared into a variety of the following forms for oral or parenteral administration when the pharmaceutical composition is clinically administered, but the present invention is not limited thereto.

For example, the formulation for oral administration includes a tablet, a pill, a hard/soft capsule, a solution, a suspension, an emulsion, a syrup, a granule, an elixir, and the like. In addition to the active ingredient, these formulations may contain a diluent (e.g., lactose, dextrose, sucrose, mannitol, sorbitol, cellulose and/or glycine), a lubricant (e.g., silica, talc, stearic acid, and a magnesium or calcium salt thereof, and/or polyethylene glycol), and the like. The tablet may also include a binder such as magnesium aluminum silicate, starch paste, gelatin, methylcellulose, sodium carboxymethylcellulose, and/or polyvinylpyrrolidine, and may optionally include a disintegrating agent such as starch, agar, alginic acid, or a sodium salt thereof or boiling mixtures, and/or an absorbent, a coloring agent, a flavoring agent, and a sweeting agent.

Also, the parenteral administration may be performed using a method such as subcutaneous injection, intravenous injection, intramuscular injection, intrathoracic injection, or dermal administration. For topical application, the composition may be formulated into an ointment or a cream, and may be prepared into a solution or a suspension by mixing the compound represented by Formula 1 or a pharmaceutically acceptable salt thereof with water as well as a stabilizing agent or a buffer. In this case, the solution or suspension may be prepared in the form of a unit dosage ampoule or vial. The composition may be sterilized or contain adjuvants such as a preservative, stabilizing agent, hydrating agent, or an emulsion promoting agent, a salt and/or buffer for control of osmotic pressure, and other therapeutically useful materials. In this case, the composition may be prepared using a conventional method such as a mixing, granulating, or coating method.

The pharmaceutical composition according to the present invention is administered at a pharmaceutically effective amount. In the present invention, the term “pharmaceutically effective amount” refers to an amount sufficient to treat a disease at a reasonable benefit/risk ratio applicable to any medical treatment. In this case, a level of the effective dose may be determined depending on the type and severity of a patient's disease, the activity of a drug, the sensitivity to the drug, an administration time, a route of administration, and a secretion rate, a therapeutic period, factors including drugs to be used together, and other factors well known in the field of medicine. The pharmaceutical composition of the present invention may be administered as an individual therapeutic agent, or may be administered in combination with other therapeutic agents. In this case, the pharmaceutical composition may be administered sequentially or concurrently with conventional therapeutic agents, and may be administered in a single dose or a multiple dose. By considering all the above factors, it is important to administer the composition at a dose in which the maximum effect can be achieved without any side effects when administered at a minimum dose. Thus, the dose of the pharmaceutical composition may be easily determined by those skilled in the related art.

Specifically, the effective dose of the pharmaceutical composition according to the present invention may vary depending on the age, sex, condition and weight of a patient, the rates of in vivo absorption, inactivation and secretion of an active ingredient, the type of a disease, and drugs to be used together. In general, the pharmaceutical composition may be administered at a dose of 0.001 to 150 mg/kg, preferably 0.01 to 100 mg/kg every day or every other day, or may be administered once to three times a day. However, because the dose of the pharmaceutical composition may increase or decrease depending on a route of administration, the severity of obesity, the sex, weight and weight of a patient, and the like, the dose of the pharmaceutical composition is not intended to limit the scope of the present invention in any manner.

In another aspect of the present invention, the present invention provides a method of preventing or treating autoimmune diseases, which includes administering the compound represented by Formula 1 or a pharmaceutically acceptable salt thereof to a subject.

In the present invention, the term “subject” refers to a target in need of treatment of a disease, and more particularly, to a mammal such as a human or a non-human primate, a mouse, a rat, a dog, a cat, a horse, and cattle.

In still another aspect of the present invention, the present invention provides a health functional food composition for preventing or improving autoimmune diseases, which includes the compound represented by Formula 1 or a pharmaceutically acceptable salt thereof. In the present invention, the compound represented by Formula 1 may be easily used to prepare health functional foods having an effect of preventing or improving autoimmune diseases, for example, as main and minor ingredients for foods, food additives, functional foods or drinks.

In the present invention, the “health functional food” refers to a group of foods, which are obtained by imparting a food with an added value by means of physical, biochemical, and bioengineering techniques so that the corresponding food can act as and express a food function according to a specific purpose, or a group of processed foods which are designed to sufficiently express in vivo regulatory functions in the body by regulating the biological defense rhythms exerted by food compositions, preventing diseases, or helping a person recover from diseases, and the like. The functional food may further include a sitologically acceptable food supplement additive, and may further include other suitable additives, such as a carrier, an excipient, and a diluent, which are commonly used in the art.

Further, the health functional food composition of the present invention may contain various nutrients, vitamins, minerals (electrolytes), flavoring agents such as synthetic and natural flavoring agents, coloring agents, and fillers (cheese, chocolate, and the like), pectic acid and salts thereof, alginic acid and salts thereof, organic acids, protective colloidal thickening agents, pH control agents, stabilizing agents, preservatives, glycerin, alcohols, carbonating agents used in carbonated drinks, and the like. In this case, the aforementioned components may be used alone or in combination.

### MODE FOR INVENTION

Hereinafter, preferred Examples are provided to aid in understanding the present invention. However, it should be understood that detailed description provided herein is merely intended to provide a better understanding of the present invention and is not intended to limit the scope of the present invention.

### EXAMPLES

### Example 1. Preparation of Compound

### 1-1. Preparation of 1-methyl-3-(4-(pyridin-2-yl)benzyl)-thiourea (JC1-38)

4-(2-pyridyl)benzaldehyde (97%, 1,000 mg, 5.46 mmol) and N-methylthiourea (4,921 mg, 54.6 mmol) were added to two round-bottom flasks, and the flasks were purged with argon gas under reduced pressure. Thereafter, anhydrous tetrahydrofuran (THF; 20 mL) was added thereto as a solvent, and Ti(OiPr)₄ (2.72 mL, 9.28 mmol), which had been kept in a freezer, was added thereto to reflux. When a substrate was completely removed on TLC, a reaction container was slowly cooled, and sodium borohydride (103 mg, 2.73 mmol) was then added thereto. Accordingly, a desired yellow product (512.9 mg, 2 mmol) was obtained (yield: 37%).

¹H-NMR (300 MHZ, CDCl₃) δ8.67-8.65 (d, J=5.0 Hz, 1H), 7.95-7.92 (d, J=8.2 Hz, 2H), 7.78-7.68 (m, 2H), 7.42-7.39 (d, J=8.0 Hz, 2H), 7.21 (s, 1H), 4.71 (s, 2H), 2.99 (s, 3H)

### 1-2. Preparation of 1-(4-benzyloxy-benzyl)-3-methyl-thiourea (JC1-40)

Triethylamine (TEA; 1.2 eq) and 4-benzyloxybenzaldehyde were added to methylamine hydrogen chloride salt (1 eq) under a DMF solvent, and then stirred. After it was confirmed that a substrate was removed on TLC, DMF was evaporated. Thereafter, the mixture was diluted with ethyl acetate, washed with brine, and then evaporated under reduced pressure to obtain a residue. The residue was subjected to column chromatography (hexane:ethyl acetate=3:1) to obtain a yellow solid (202.7 mg, 0.71 mmol) (yield: 30%).

¹H-NMR (300 MHZ, CD₃OD) δ7.44-7.28 (m, 5H), 7.23-7.20 (d, J=8.4 Hz, 2H), 6.98-6.94 (m, 2H), 5.07 (s, 2H), 4.55 (s, 2H), 2.82 (s, 3H)

### 1-3. Preparation of 1-(4-phenoxy-benzyl)-3-methyl-thiourea (JC1-42)

Triethylamine (TEA; 1.2 eq) and 4-phenoxybenzaldehyde were added to methylamine hydrogen chloride salt (1 eq) under a DMF solvent, and then stirred. After it was confirmed that a substrate was removed on TLC, DMF was evaporated. Thereafter, the mixture was diluted with ethyl acetate, washed with brine, and then evaporated under reduced pressure to obtain a residue. The residue was subjected to column chromatography (hexane:ethyl acetate=3:1) to obtain a yellow solid (178.9 mg, 0.66 mmol) (yield: 65%).

¹H-NMR (300 MHZ, CDCl₃) δ7.33-7.23 (m, 4H), 7.11-7.06 (m, 1H), 6.98-6.92 (m, 4H), 4.62 (s, 2H), 2.96-2.94 (d, J=4.6 Hz, 3H)

### Example 2. Confirmation of Inhibitory Effect of Thiourea Derivative on Transcription of Inflammatory Genes in Macrophage Cell Line

A mouse macrophage cell line Raw 264.7 (ATCC TIB-71) was purchased from the American Type Culture Collection (ATCC). Raw 264.7 cells (2×10⁵ cells/well) were seeded in a 6-well culture dish, and cultured for a day in a Dulbecco's modified Eagle's medium (DMEM) supplemented with 10% fetal bovine serum (FBS). The Raw 264.7 cells were kept at 37° C. in a thermo-hygrostat chamber in which 5% CO₂ and 95% air were maintained. After the culturing, the cells were treated with 50 ng/mL of lipopolysaccharides (LPS) and 20 μM JC1-40 for 24 hours. Then, mRNA expression levels of the inflammatory markers, such as TNF-α, NOS2, IL-1β, and IL-6, which induce rheumatoid arthritis, were measured using a real-time PCR (i.e., real-time reverse transcriptase-polymerase chain reaction; quantitative polymerase chain reaction) assay.

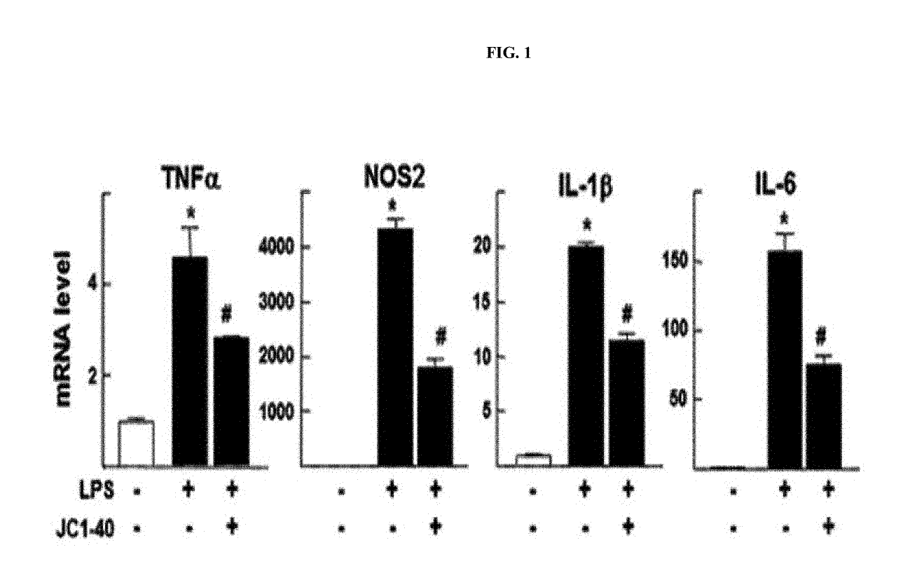

As a result, it can be seen that the transcription levels of TNF-α, NOS2, IL-1β and IL-6, which are involved in the induction of inflammation, in the Raw 264.7 macrophage cell line were reduced when the cells were treated with JC1-40, as shown in FIG. 1.

### Example 3. Confirmation of Inhibitory Effect of Thiourea Derivative on Transcription of Inflammatory Genes in Primarily Cultured Mouse Intraperitoneal Macrophages

Peritoneal macrophages of 7 to 8-week-old C57BL/6 male mice were isolated, seeded in a 24-well dish at a density of 1×10⁶ cells/well, and then cultured for 4 hours in an RPMI1640 medium supplemented with 10% fetal bovine serum (FBS). A peritoneal macrophage culture broth was treated with each of 10 ng/mL of LPS and 20 μM JC1-40 or JC1-42, as indicated herein, for 24 hours. In this case, the peritoneal macrophages were kept at 37° C. in a thermo-hygrostat chamber in which 5% CO₂ and 95% air were maintained.

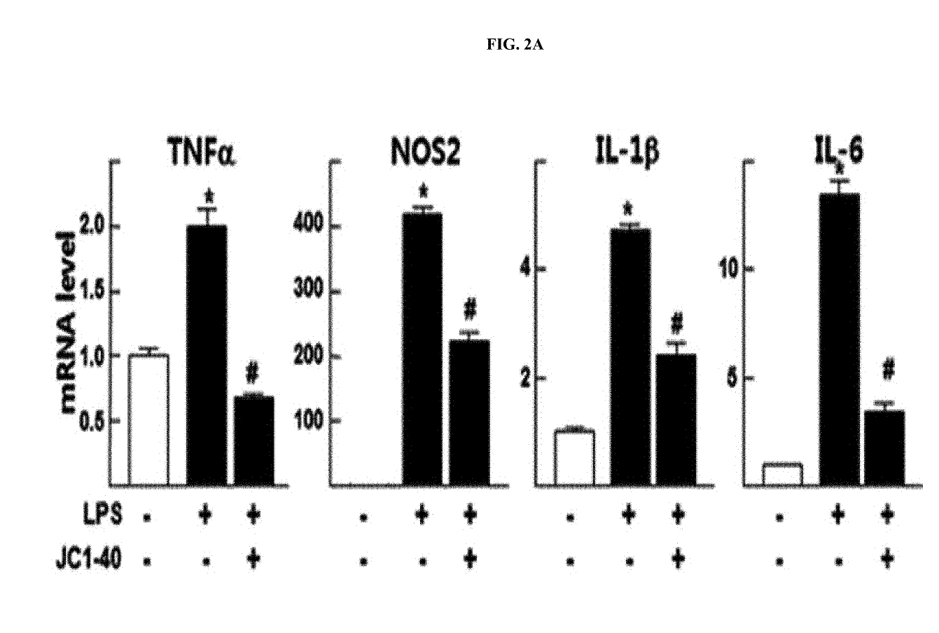

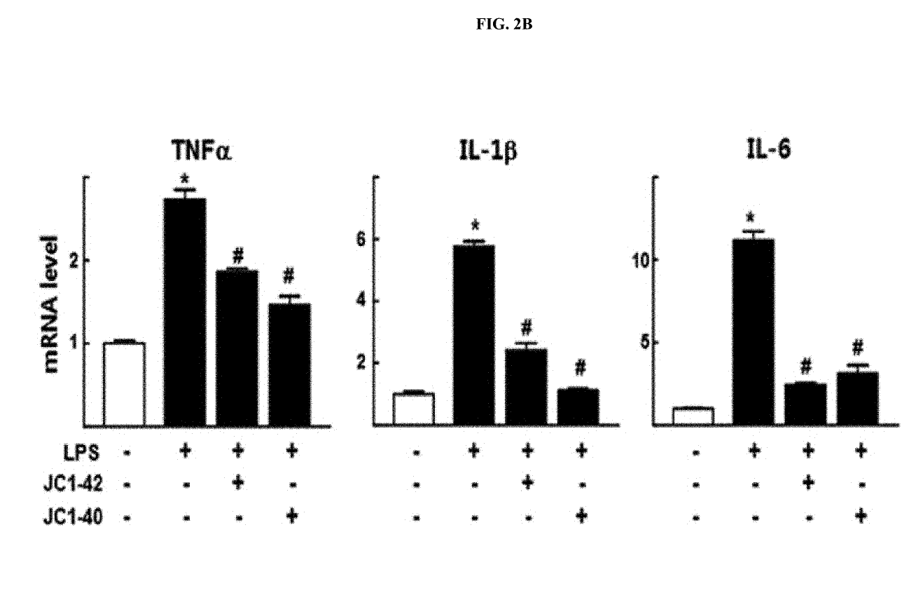

First, mRNA expression levels of the inflammatory markers, such as TNF-α, NOS2, IL-1β, and IL-6, which induce rheumatoid arthritis, were measured using a real-time PCR (i.e., real-time reverse transcriptase-polymerase chain reaction; quantitative polymerase chain reaction) assay. As a result, it can be seen that the mRNA levels of TNF-α, NOS2, IL-1β and IL-6, which are involved in the induction of inflammation, in the intraperitoneal macrophages were reduced when the cells were treated with JC1-40 or JC1-42, as shown in FIG. 2.

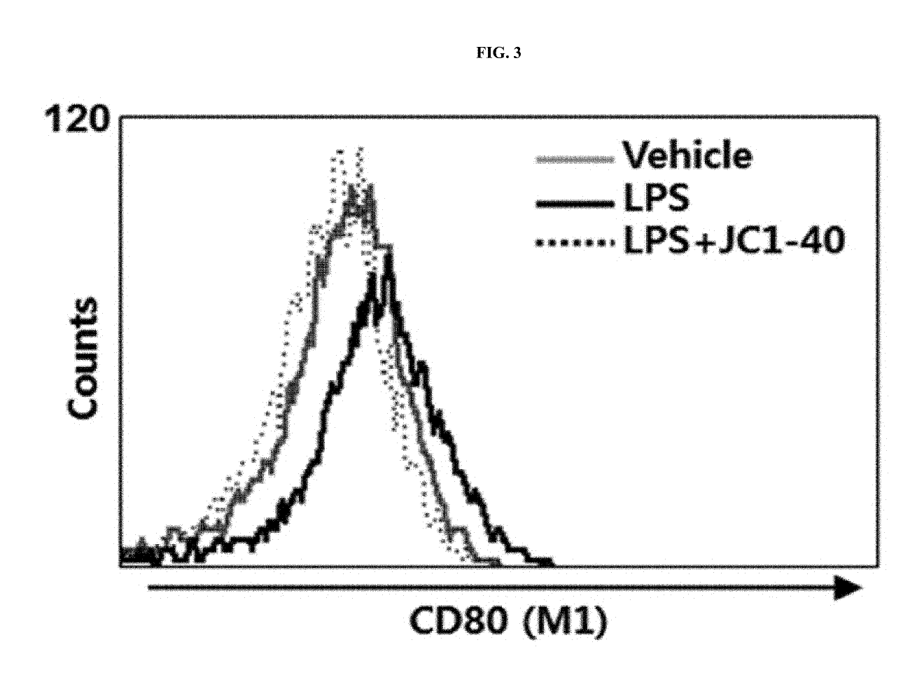

Further, a PE-CD80 antibody (eBioscience), which is a M1 marker for recognizing a change of M1 cells, was incubated in these cells for 30 minutes, and then subjected to flow cytometry. As a result, it was confirmed that the M1 differentiation was suppressed when the macrophages were treated with JC1-40, as shown in FIG. 3.

### Example 4. Confirmation of Promotion Effect of Thiourea Derivative on Transcription of M2 Anti-Inflammatory Genes in Primarily Cultured Mouse Intraperitoneal Macrophages

Peritoneal macrophages of 7 to 8-week-old C57BL/6 male mice were isolated, seeded in a 24-well dish at a density of 1×10⁶ cells/well, and then cultured for 4 hours in an RPMI1640 medium supplemented with 10% fetal bovine serum (FBS). A peritoneal macrophage culture broth was treated with each of 20 ng/mL of IL-4 and 20 μM JC1-40 or JC1-42, as indicated herein, for 24 hours. Thereafter, mRNA expression levels of the M2 anti-inflammatory markers, such as IL-10, Arg1, Retnla, and CD206, which induce rheumatoid arthritis, were measured using a real-time PCR (i.e., real-time reverse transcriptase-polymerase chain reaction; quantitative polymerase chain reaction) assay. In this case, the peritoneal macrophages were kept at 37° C. in a thermo-hygrostat chamber in which 5% CO₂ and 95% air were maintained.

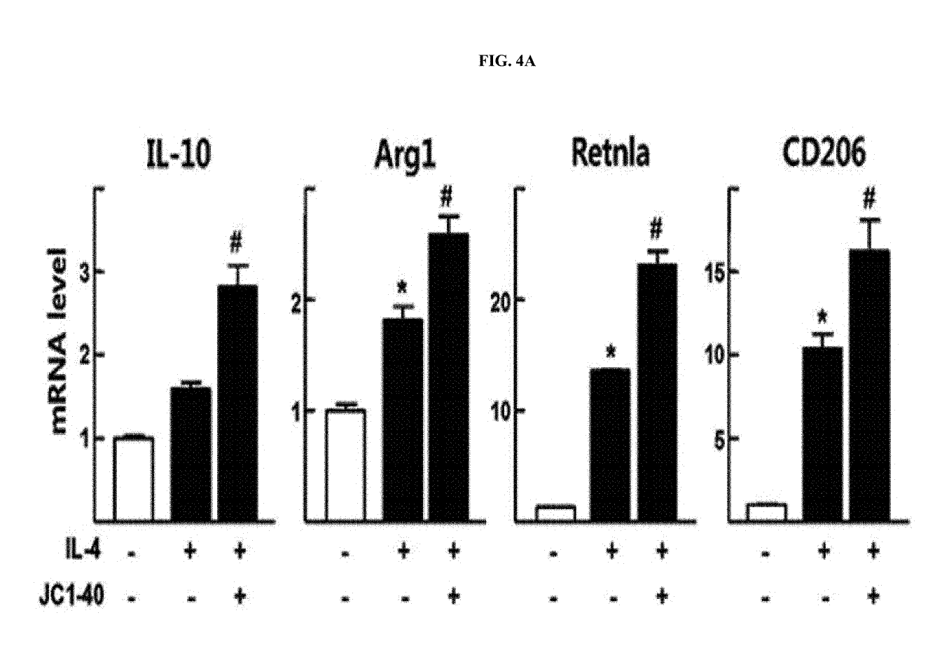

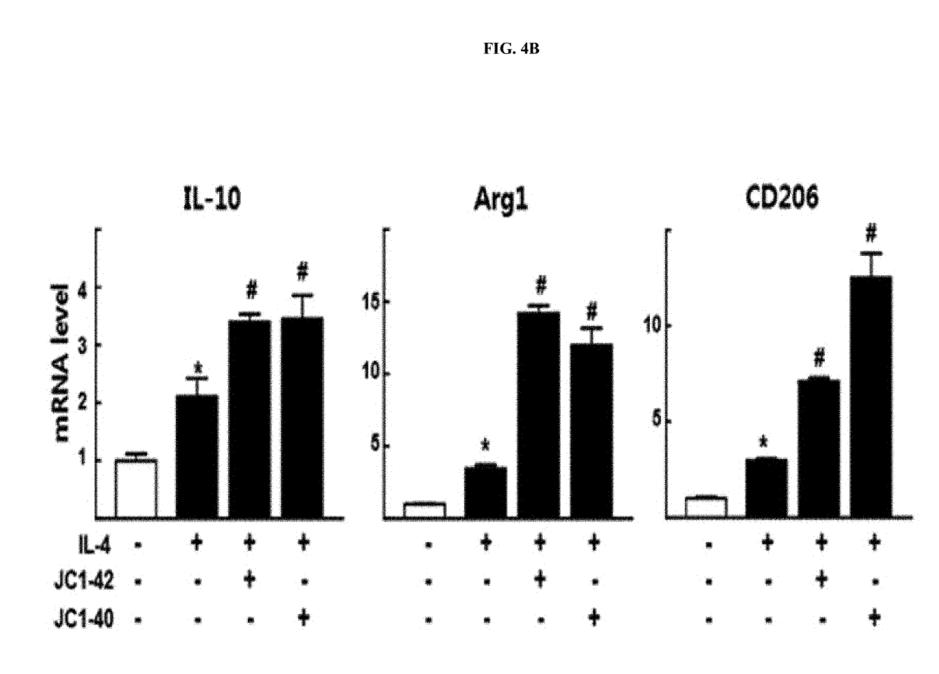

As a result, it can be seen that the transcription levels of IL-10, Arg1, Retnla and CD206, which are involved in the anti-inflammatory regulation, was increased in the intraperitoneal macrophage cell line when the cell line was treated with JC1-40 or JC1-42, as shown in FIGS. 4A and 4B.

### Example 5. Confirmation of Inhibitory Effect of Thiourea Derivative on Th17 Differentiation and IL-17 Secretion in T Cell Line

A mouse T cell EL4 cell line (5×10⁵ cells/well) was seeded in a 24-well culture dish, pre-treated with 20 μM JC1-40 for 24 hours, and cultured for a day in a RPMI 1640 medium supplemented with 10% FBS. The EL4 cell line was kept at 37° C. in a thermo-hygrostat chamber in which 5% CO₂ and 95% air were maintained. After the culturing, the cells were cultured for 48 hours under Th17 differentiation conditions (including 0.5 μg/mL of an anti-CD3 antibody, 1 μg/mL of an anti-CD28 antibody, 2 ng/mL of TGF-β, 20 ng/mL of IL-6, 10 μg/mL of anti-IL-4, and 10 μg/mL of anti-IFN-γ). Expression levels of RORα, which is a JC1-40-activated nuclear receptor, and IL-17, which is an inflammatory marker inducing rheumatoid arthritis, were measured using a real-time PCR (i.e., real-time reverse transcriptase-polymerase chain reaction; quantitative polymerase chain reaction) assay.

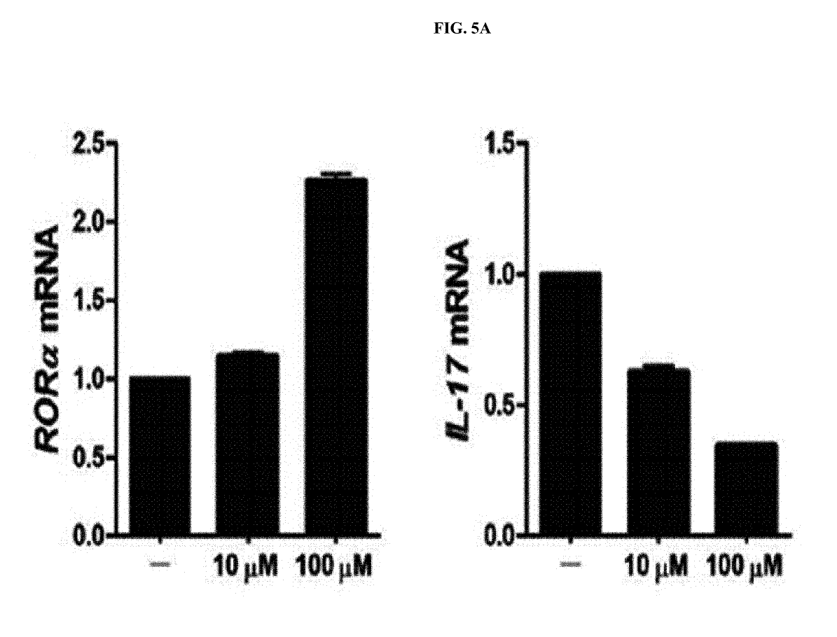

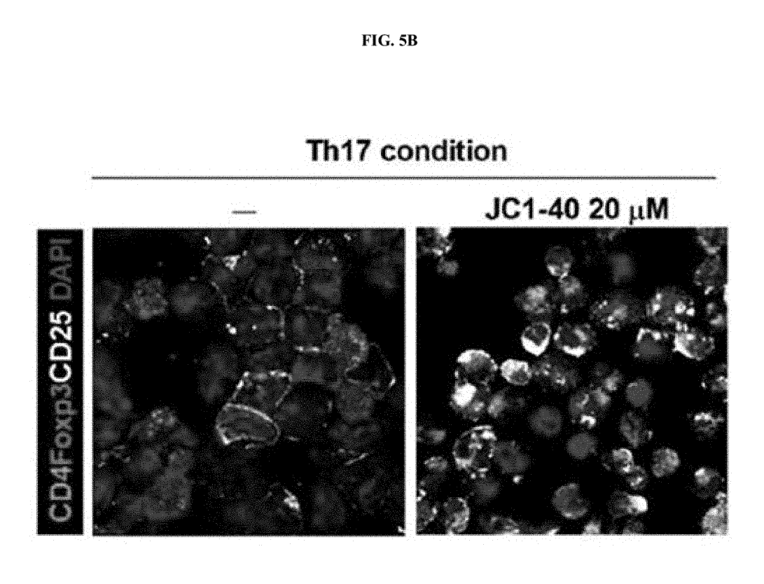

As a result, it can be seen that the transcription level of the nuclear receptor (i.e., RORα) increased and the transcription level of IL-17 decreased when the EL4 cell line was treated with JC1-40, as shown in FIG. 5A. Also, the fact that an expression level of Foxp3, which is a representative factor in Tregs which suppress Th17 activity, increased by means of JC1-40 treatment was confirmed through confocal microscopy observation after the cells cultured under the aforementioned conditions were stained with fluorescence-labeled antibodies (anti-CD4-FITC, anti-CD25-APC, and anti-Foxp3-PE) (see FIG. 5B).

### Example 6. Confirmation of Inhibitory Effect of Thiourea Derivative on Th17 Differentiation and IL-17 Secretion in Primarily Cultured Mouse Spleen T Cells

CD4+ T cells were isolated from the spleens of 7 to 8-week-old C57BL/6 male mice, seeded in a 48-well dish at a density of 5×10⁵ cells/well, pre-treated with a varying concentration of JC1-40 (0.1, 0.5, 10, and 50 μM) for 24 hours, and then cultured for a day in an RPMI1640 medium supplemented with 10% fetal bovine serum (FBS). After the culturing, the cells were cultured for 3 days under Th17 differentiation conditions (including 0.5 μg/mL of an anti-CD3 antibody, 1 μg/mL of an anti-CD28 antibody, 2 ng/mL of TGF-β, 20 ng/mL of IL-6, 10 μg/mL of anti-IL-4, and 10 μg/mL of anti-IFN-γ). Thereafter, the cells were collected, washed with a FACS buffer for flow cytometry, and then blocked at 4° C. for 15 minutes to inhibit non-specific binding. As a cell surface marker, an antibody against CD4 (anti-CD4-PerCP) was added thereto, and reacted at 4° C. for 30 minutes, and the resulting reaction mixture was washed with a perm wash buffer. After a Cytofix/Cytoperm process was performed at 4° C. for 20 minutes, the mixture was washed with a perm wash buffer. Anti-IL-17 PE was added thereto, and reacted at 4° C. for 30 minutes, and the resulting reaction mixture was washed with a perm wash buffer. A change of the stained cells was analyzed using flow cytometry.

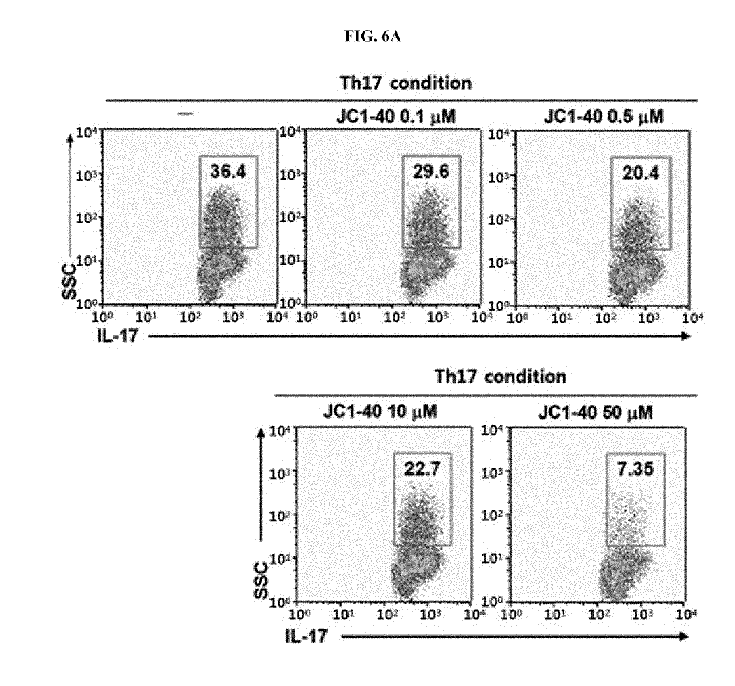

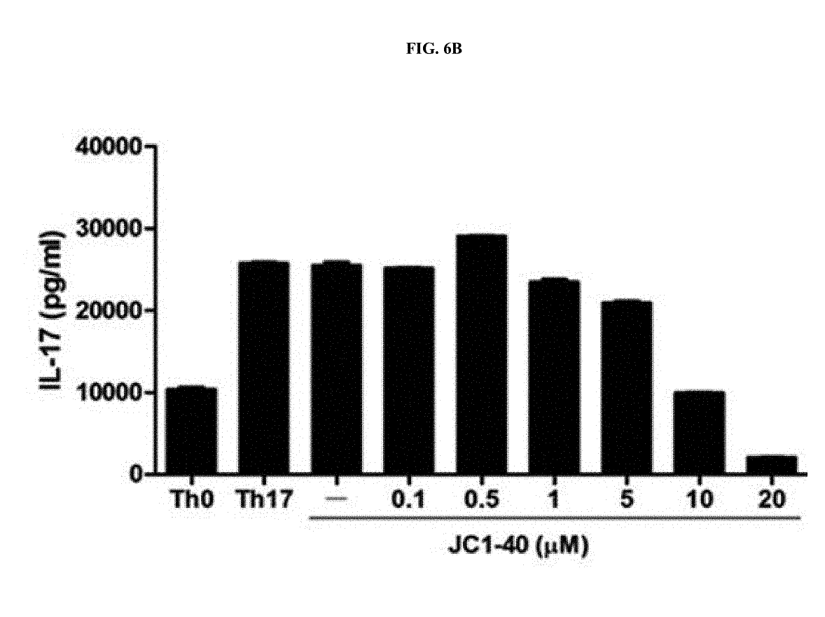

As a result, it can be seen that an amount of IL-17 positive cells differentiated into Th17 decreased when the cells were treated with JC1-40, as shown in FIG. 6A.

In addition, a culture supernatant of the JC1-40-treated Th17 cells was collected to determine an amount of IL-17 using a sandwich ELISA method. For this purpose, first of all, a 96-well plate was treated with monoclonal anti-IL-17 at a concentration of 2 μg/mL, and reacted overnight at 4° C. After the reaction, the reaction solution was blocked with a blocking solution (1% BSA/PBST) to prevent non-specific binding. Thereafter, IL-17 was continuously diluted two-fold, and used as the standard. The cell culture supernatant was added thereto, and reacted at room temperature for 2 hours. Subsequently, biotinylated anti-IL-17 was reacted therein at room temperature for 2 hours, and the resulting reaction mixture was washed four times. An ExtraAvidin-Alkaline Phosphatase conjugate was diluted and added thereto, and reacted at room temperature for 2 hours. Then, a PNPP/DEA solution was added thereto, and developed color, and the optical density was then measured at a wavelength of 405 nm.

As a result, it can be seen that an amount of the secreted IL-17 protein in the culture broth was reduced when the cells were treated with JC1-40, as shown in FIG. 6B.

### Example 7. Confirmation of Inhibitory Effect of Thiourea Derivative on Th17 Differentiation and IL-17 Secretion in Human Peripheral Blood Mononuclear Cells (PBMCs)

CD4+ cells were isolated from peripheral blood mononuclear cells (PBMCs) of a healthy human, pre-treated with a varying concentration (10, 20, and 40 μM) of JC1-40, and then cultured for 3 days under Th17 differentiation conditions (including 0.5 μg/mL of anti-CD3, 0.5 μg/mL of anti-CD28, 10 μg/mL of anti-IFN-γ, 10 μg/mL of anti-IL-4, 20 ng/mL of IL-6, and 20 ng/mL of IL-1β). The expression of Th17 (anti IL-17 PE) and Treg (Anti Foxp3-FITC) in these cells was confirmed using flow cytometry. Also, an amount of IL-17 in these supernatants was measured using ELISA.

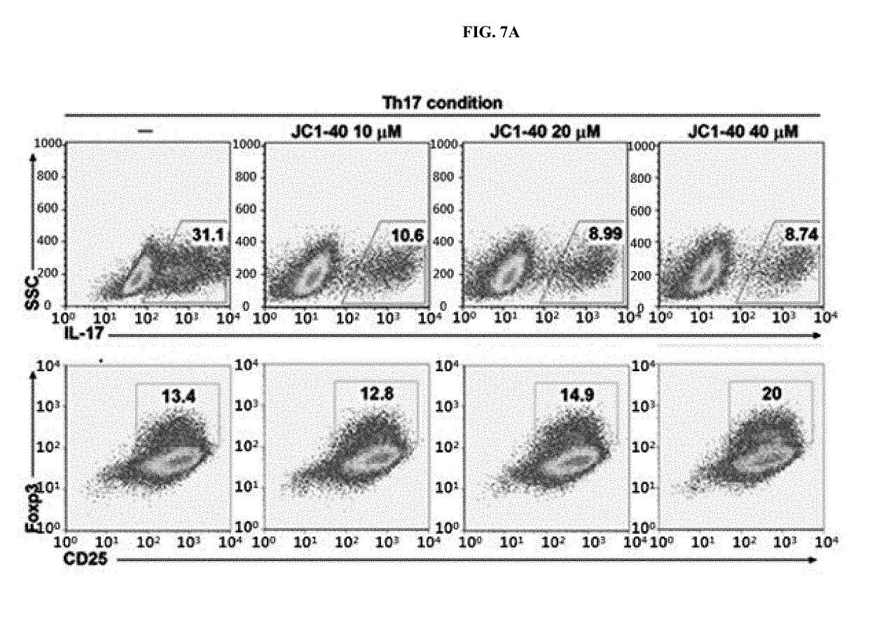

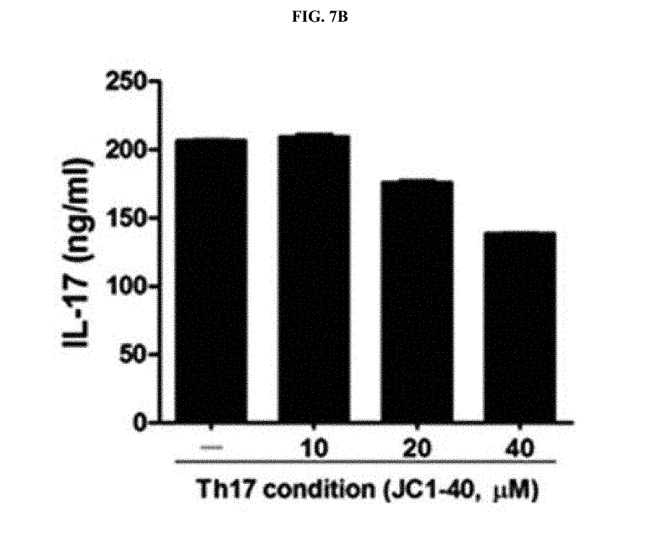

As a result, it can be seen that an amount of the (IL-17 positive) T cells differentiated into Th17 decreased but an amount of the (FOXP3 positive) cells differentiated into Treg increased when the human peripheral blood mononuclear cells were treated with JC1-40, as shown in FIGS. 7A and 7B.

### Example 8. Confirmation of Regulatory Effect of Thiourea Derivative on Osteoclast Differentiation

PBMCs of a healthy human were isolated, stimulated with M-CSF (25 ng/mL) for 3 days to induce differentiation into an osteoclast precursor. After 3 days, a medium was replaced with a fresh one, and the cells were stimulated with varying concentrations of M-CSF (25 ng/mL), RANKL (30 ng/mL), and JC1-40 (10, 20, 40 μM), and cultured for 3 days. After the medium was replaced every 3 days, the same stimulus was applied to the cells while a differentiation state of the cells was observed.

RNA was isolated from the differentiated cells, and synthesized into cDNA. Thereafter, a change in mRNA expression levels of the osteoclast differentiation-related factors, Cathepsin K and TRAP, was analyzed using real-time PCR.

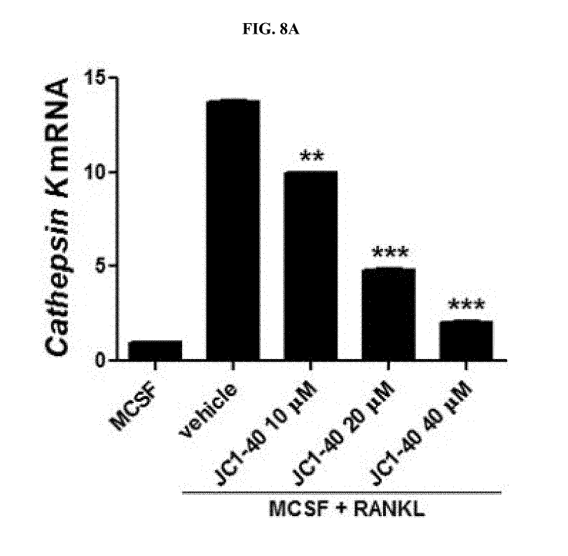

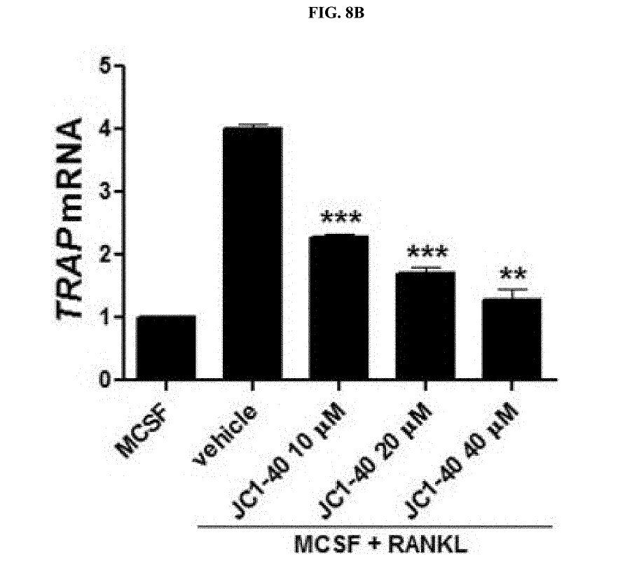

As a result, it can be seen that the expression of the osteoclast differentiation-related factors was significantly reduced when the cells were treated with JC1-40, as shown in FIGS. 8A and 8B.

### Example 9. Confirmation of Inhibitory Effect of Thiourea Derivative on Arthritis in Arthritis Animal Model (Type II Collagen-Induced Arthritis (CIA))

6-week-old DBA male mice were inoculated with bovine CII (Chondrex Inc.) emulsified in CFA (Chondrex Inc.), and re-inoculated after 15 days to induce rheumatoid arthritis. After the first administration, JC1-40 dissolved in 0.5% carboxymethyl cellulose (CMC) was continuously orally administered three times a week to the mice at a dose of 10 mg/kg. A 0.5% CMC vehicle and 5 mg/kg of Enbrel as the positive control were administered to the other groups in the same manner. The ratio of the population of the mice developing an arthritic disease was calculated as incidence, and the arthritic tissues were pathophysiologically examined to calculate a degree of progression of the disease as a score and compare conditions of the rheumatoid arthritis disease between the groups. The clinical scores were evaluated using a clinical scoring system which scores the swelling and redness in feet or tail regions by observing arthritis lesions of mice with the naked eye every day to check a degree of progression of inflammatory diseases in the joints.

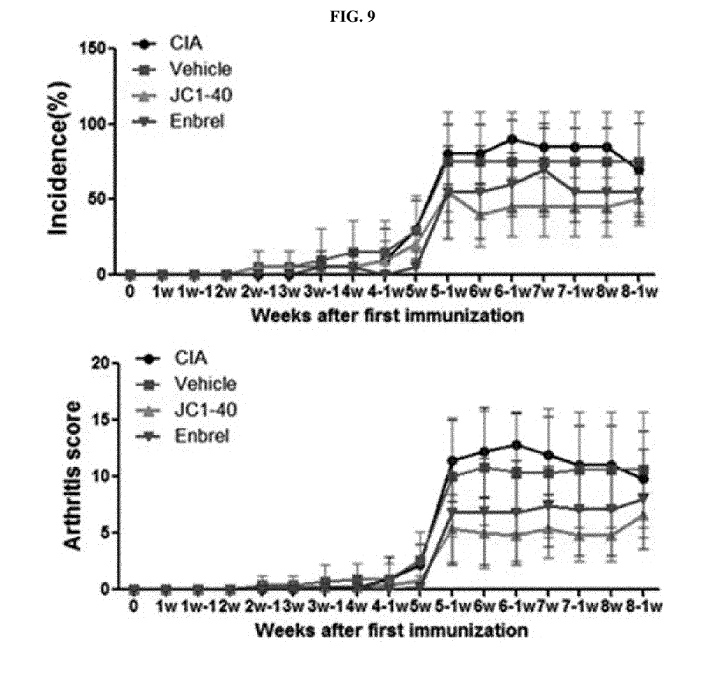

As a result, it was confirmed that the JC1-40 administration in the CIA animal model suppressed both arthritis incidence and scores, as shown in FIG. 9, indicating that JC1-40 had a superior effect, compared to Enbrel which is a therapeutic agent for rheumatoid arthritis actually used in clinical practice.

Although the present invention presented herein has been disclosed for illustrative purposes, it should be apparent to those skilled in the art to which the present invention belongs that various modifications and changes are possible without departing from the scope and spirit of the present invention. Therefore, it should be understood that the exemplary embodiments disclosed above are illustrative in all aspects and are not intended to limit the present invention.

### INDUSTRIAL APPLICABILITY

The thiourea derivative according to the present invention can inhibit the transcriptions of inflammatory genes, promote the transcription of anti-inflammatory genes, and simultaneously inhibit the activation or production of Th17 cells and increase the activation or production of regulatory T cells (Tregs). Therefore, the thiourea derivative according to the present invention can be expected to be effectively used in a pharmaceutical composition, a health food composition, and the like for preventing, improving or treating various autoimmune diseases including rheumatoid arthritis.

1. A method of treating an autoimmune disease, comprising administering to a subject in need thereof a pharmaceutical composition comprising an effective amount of a compound represented by the following Formula 1 or a pharmaceutically acceptable salt thereof: 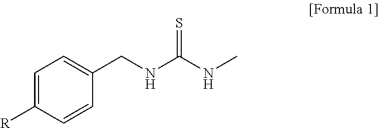
wherein R represents a phenoxy group, a benzyloxy group, or a pyridinyl group.

2. The method of claim 1, wherein the autoimmune disease is selected from the group consisting of rheumatoid arthritis, psoriatic arthritis, psoriasis, systemic scleroderma, sclerosis, multiple sclerosis, inflammatory bowel disease, Crohn's disease, septicemia, and type I diabetes.

3. The method of claim 1, wherein the compound inhibits the production of Th17 cells.

4. The method of claim 1, wherein the compound promotes the production of regulatory T cells (Tregs).

5. The method of claim 1, wherein the compound inhibits the expression of a TNF-α, NOS2, IL-1β or IL-6 gene.

6. The method of claim 1, wherein the compound promotes the expression of an IL-10, Arg1, Retnla or CD206 gene.

7.-8. (canceled)
